(19) United States
(12) Patent Application Publication
(10) Pub. No.: US 2025/0223569 A1
(43) Pub. Date: Jul. 10, 2025

(54) INITIALIZATION AGENT AND KIT FOR INITIALIZING BLOOD CELLS DERIVED FROM PERIPHERAL BLOOD, AND METHOD FOR INITIALIZING THE SAME

(71) Applicants: KABUSHIKI KAISHA TOSHIBA, Tokyo (JP); CiRA Foundation, Kyoto-shi (JP)

(72) Inventors: Emi NOZAKI, Shinagawa (JP); Mitsuko ISHIHARA, Setagaya (JP); Masafumi UMEKAGE, Kyoto-shi (JP)

(73) Assignees: KABUSHIKI KAISHA TOSHIBA, Tokyo (JP); CiRA Foundation, Kyoto-shi (JP)

(21) Appl. No.: 18/999,964

(22) Filed: Dec. 23, 2024

(30) Foreign Application Priority Data
Jan. 5, 2024 (JP) 2024-000721

(51) Int. Cl. C12N 5/074; C12N 15/113; C12N 15/88

(52) U.S. Cl. CPC C12N 5/0696; C12N 15/113; C12N 15/88; C12N 2310/141; C12N 2501/602; C12N 2501/603; C12N 2501/604; C12N 2501/605; C12N 2501/606; C12N 2501/608; C12N 2506/11; C12N 2510/00

(57) ABSTRACT

According to one embodiment, an initialization agent is for initializing blood cells derived from peripheral blood. The initialization agent includes an initialization factor group for producing iPS cells by initializing a group of blood cells including mononuclear cells derived from peripheral blood, and a group of lipid nanoparticles that encapsulate the initialization factor group. The lipid nanoparticles have a component of at least or more of 40% FFT-10 and FFT-20. FFT-10 and FFT-20 are included in equal amounts.

### CROSS-REFERENCE TO RELATED APPLICATIONS

This application is based upon and claims the benefit of priority from Japanese Patent Application No. 2024-000721, filed Jan. 5, 2024, the entire contents of which are incorporated herein by reference.

### FIELD

Embodiments described herein relate generally to an initialization agent and kit for initializing blood cells derived from peripheral blood, and to a method for initializing the same.

### BACKGROUND

Induced pluripotent stem cells, generally referred to as iPS cells, are produced using a viral vector by making the cell to express an initialization factor. When using a viral vector, there is a risk that the vector will remain in the infected cell and the cell continues to express the initialization factors. There is also a further risk that the virus may remain in the final product. On the other hand, there is also a method that uses lipofection, but it is not easy to stably or homogeneously produce iPS cells when using this method.

### BRIEF DESCRIPTION OF THE DRAWINGS

FIG. 1 is a cross-sectional view showing an example of an initialization agent according to the first embodiment.

FIG. 2 is a cross-sectional view showing another example of the initialization agent according to the first embodiment.

FIG. 3 is a cross-sectional view showing still another example of the initialization agent according to the first embodiment.

FIG. 4 is a schematic diagram showing a method according to the third embodiment.

FIG. 5 is a diagram showing experimental results.

FIG. 6 is a diagram showing images of the experimental results.

FIG. 7 is a diagram showing images of the experimental results.

FIG. 8 is a diagram showing images of the experimental results.

### DETAILED DESCRIPTION

In general, according to one embodiment, an initialization agent is for initializing blood cells derived from peripheral blood. The initialization agent includes an initialization factor group for producing iPS cells by initializing a group of blood cells including mononuclear cells derived from peripheral blood, and a group of lipid nanoparticles that encapsulate the initialization factor group. The lipid nanoparticles have a component of at least or more of 40% FFT-10 and FFT-20. FFT-10 and FFT-20 are included in equal amounts.

Embodiments will now be described with reference to the accompanying drawings. In these embodiments, substantially identical structural elements are denoted by the same symbols, and some of the descriptions may be omitted. Note that the drawings are schematic, and the relationship between the thickness and planar dimensions of each part, the ratio of the thickness of each part, etc., may differ from the actual ones.

### First Embodiment

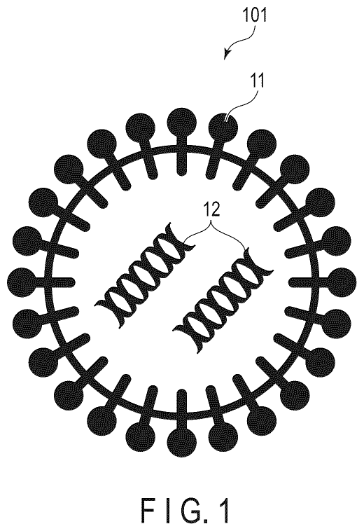

The initialization agent according to the embodiment is for initializing blood cells derived from peripheral blood. The initialization agent comprises a group of initialization factors for initializing a group of blood cells including mononuclear cells derived from peripheral blood to produce iPS cells, and a group of lipid nanoparticles that encapsulate the initialization factors. The lipid nanoparticles described above contain at least 40% or more of FFT-10 and FFT-20 of the entire component. FFT-10 and FFT-20 are contained in equal amounts. An example of the structure thereof is shown in FIG. 1. The initialization agent may include, for example, a nucleic acid introduction carrier. A nucleic acid introduction carrier 101 comprises lipid nanoparticles 11 creating a hollow portion and an initialization factors group 12 encapsulated therein. The initialization factor group 12 is a group of initialization factors for initializing a group of blood cells, including mononuclear cells derived from peripheral blood, so as to produce iPS cells.

The initialization factor group 12 is a factor that initializes mononuclear cells and creates iPS cells when the nucleic acid introduction carrier 101 is added to a group of blood cells including mononuclear cells derived from peripheral blood. For example, the initialization factor 12 can be any factor that can initialize and reprogram the DNA information in the nucleus. For example, the initialization factor is mRNA, in which case, the initialization factor is introduced into the cytoplasm, remains in the cytoplasm, and is translated into transcription factor, which is a protein as an initialization substance, and reprograms the DNA in the nucleus. The number of types of initialization factor may be one or two or more. The required reprogramming factors may be introduced by a nucleic acid introduction carrier 101 according to the state of the cells and the timing of administration. Examples of the initialization factor include, but are not limited to, messenger RNA of Oct3/4, Sox2, Klf4, cMyc, Nanog, and Lin28. For example, the above-described initialization factor group may include Oct3/4, Sox2, Klf4, and cMyc. Further, the above-described initialization factor group may as well include Nanog and Lin28, and may further include these in addition to Oct3/4, Sox2, Klf4, and cMyc. The initialization factor group encapsulated in the lipid nanoparticles can be contained in the form of DNA, RNA and/or protein, for example.

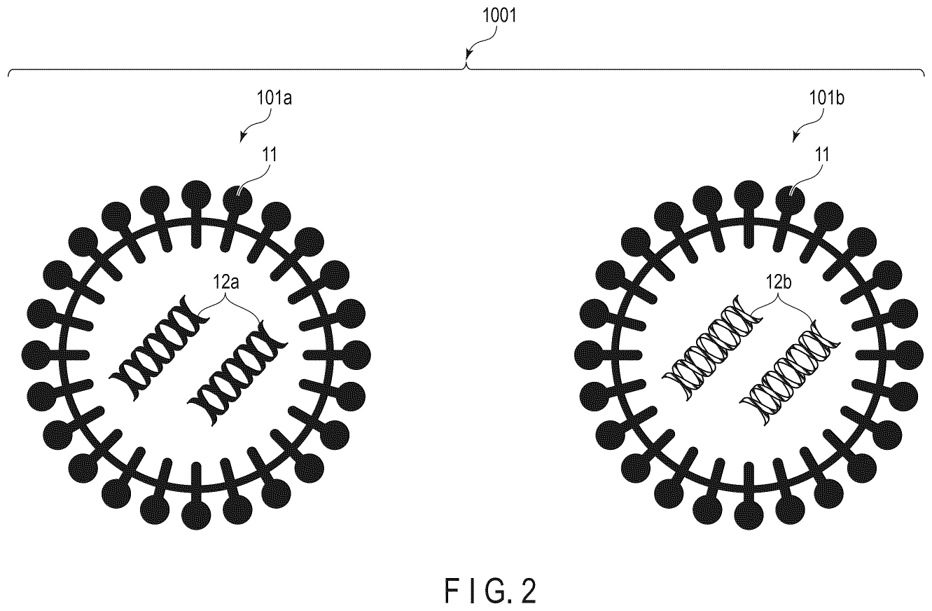

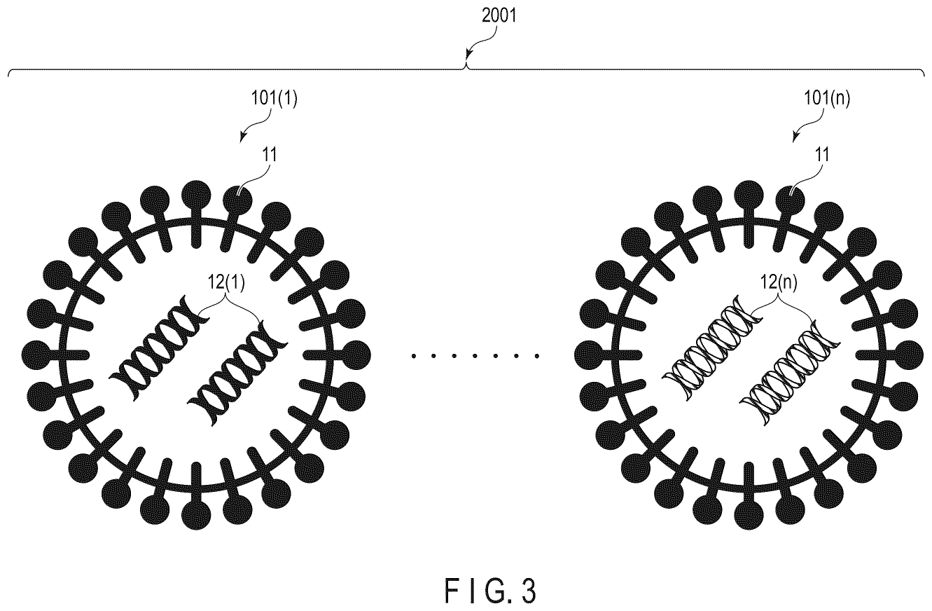

For example, the lipid nanoparticles may encapsulate initializing factor groups to be introduced in two parts, as shown in FIG. 2. In that case, the initializing agent may be provided as a nucleic acid introduction carrier set 1001 comprising a first nucleic acid introduction carrier 101a containing a first initializing factor group 12a and lipid nanoparticles 11 encapsulating the first initializing factor group 12a, and a second nucleic acid introduction carrier 101b comprising a second initializing factor group 12b and lipid nanoparticles 11 encapsulating the second initializing factor group 12b. Alternatively, as shown in FIG. 3, it may be provided as a nucleic acid introduction carrier set 2001 comprising a first nucleic acid introduction carrier 101(1) to an n-th nucleic acid introduction carrier 101(n), that is, total n-types of lipid nanoparticles 11, each of which encapsulates different types of the initialization factor 12(1) to 12(n) selected from the initialization factor group to be introduced. Here, n is an integer greater than or equal to 2. Further, a desired number of types of nucleic acid introduction carriers may be divided and contained in a desired number of types of initializing factors.

The lipid nanoparticles can be liposomes, which are particles of lipid membranes that encapsulate cores of an aqueous solution, such as particles of lipid bilayer membranes. The lipid composition which forms the liposomes includes first lipids (FFT-10) of Formula (I) and/or second lipids (FFT-20) of Formula (II) as its components. These lipids are biodegradable lipids. The lipid nanoparticles contain at least 40% or more of FFT-10 and FFT-20, and FFT-10 and FFT-20 are contained in equal amounts. With use of lipid nanoparticles having such a lipid composition, it is possible to initialize blood cells derived from peripheral blood.

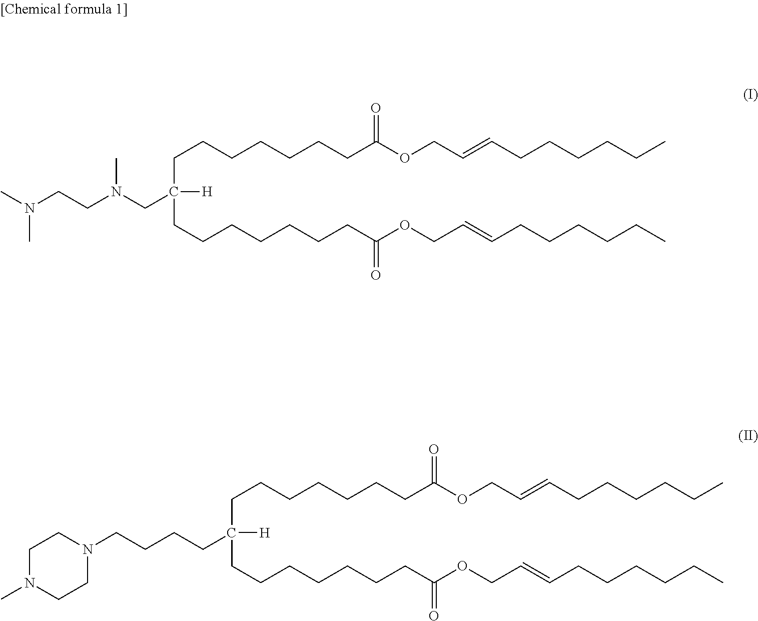

The lipid nanoparticles may contain further lipids in addition to FFT-10 and FFT-20 as the first lipids and second lipids, respectively. In the composition of the lipid molecular material that composes the lipid nanoparticles, a fraction constituted by the first lipids and second lipids is referred to as a “first fraction” hereinafter. Further, a fraction composed of lipid molecular materials other than those of the first lipids and the second lipids is referred to as a “second fraction” hereinafter. The lipids contained in the second fraction are collectively referred to as “third lipids” hereinafter.

The terms “first fraction” and “second fraction” express the type of composition of the components of the lipid nanoparticles, and do not indicate the physical location of the lipids contained therein. For example, the components of the first fraction and the second fraction do not need to be in separate groups within the lipid nanoparticles, and the lipids contained in the first fraction and the lipids contained in the second fraction can exist in a mixed state. The composition ratio of the first fraction to the total lipid material that makes up the lipid nanoparticles can be, for example, 40% or more, for example, 40% to 45%, 40% to 50%, etc. FFT-10 and FFT-20 are contained in equal amounts, for example, 17% to 23% and 17% to 23%, respectively. Note that, percentages in this context are expressed in mol % unless otherwise stated.

The type of the third lipids contained in the second fraction of the lipid nanoparticles is not particularly limited, but, for example, the second fraction contains basic lipids. As the basic lipid, for example, lipids that are a main component of a biological membrane can be used. Examples of the basic lipids are a phospholipid and a sphingolipid, such as diacyl phosphatidyl choline, diacyl phosphatidyl ethanolamine, ceramide, sphingomyelin, dihydrosphingomyelin, cephalin, or cerebroside, or a combination of any of these.

For example, as basic lipids, 1,2-dioleoyl-sn-glycero-3-phosphoethanolamine (DOPE), 1,2-stearoyl-sn-glycero-3-phosphoethanolamine (DSPE), 1,2-dipalmitoyl-sn-glycero-3-phosphatidylcholine (DPPC), 1-palmitoyl-2-oleoyl-sn-glycero-3-phosphatidylcholine (POPC), 1,2-di-O-octadecyl-3-trimethylammonium propane (DOTMA), 1,2-dioleoyl-3-dimethylammonium propane (DODAP), 1,2-dimyristoyl-3-dimethylammonium propane (14:0 DAP), 1,2-dipalmitoyl-3-dimethylammonium propane (16:0 DAP), 1,2-distearoyl-3-dimethylammonium propane (18:0 DAP), N-(4-Carboxybenzyl)-N,N-dimethyl-2,3-bis(oleoyloxy) propane (DOBAQ), 1,2-dioleoyl-3-trimethylammonium propane (DOTAP), 1,2-dioleoyl-sn-glycero-3-phosphocholine (DOPC), 1,2-Dilinoleoyl-sn-glycero-3-phosphocholine (DLPC), or 1,2-Dioleoyl-sn-glycero-3-phospho-L-serine (DOPS), or a combination of any of these can be used. As the basic lipids, cationic lipids or neutral lipids should preferably be used, and the acid dissociation constant of the lipid nanoparticles can be adjusted by the content of the lipids. It is preferable to use DOTAP as the cationic lipids and DOPE as the neutral lipids.

It is also preferable that the second fraction should contain lipids that prevent aggregation of the lipid nanoparticles. Examples of the lipids that prevent aggregation are PEG-modified lipids, such as polyethylene glycol (PEG) dimyristoyl glycerol (DMG-PEG), polyamide oligomers derived from omega-amino (oligo ethylene glycol) alkanoate monomer (U.S. Pat. No. 6,320,017 B) and monosialogangliosides.

The second fraction may further contain lipids for adjusting toxicity, which have relatively low toxicity; lipids with functional groups that bind ligands to lipid nanoparticles; and lipids for inhibiting the leakage of internal contents such as sterols, e.g., cholesterol. In particular, it is preferable to contain cholesterol.

In addition to the initializing factors, other components may be further encapsulated in the lipid nanoparticles as needed. Such further components include, for example, a pH adjusters, an osmotic pressure adjuster, and a gene activator. The pH adjuster may be an organic acids such as citric acid or a salt thereof. The osmotic pressure adjuster may be a sugar or amino acid. Here, the gene activator can be any substance that promotes or supports the activity of the initialization factor when the initialization factor is a gene. Also, the lipid nanoparticles may contain further factors other than the initialization factor group. For example, such further factor can be one that suppresses the response of interferon or miRNA that improves the efficiency of establishing iPS cells, or the like.

The lipid nanoparticles that encapsulate the initialization factors and other substances as needed can be produced, for example, using known methods used for encapsulating small molecules in lipid nanoparticles, such as the Bangham method, organic solvent extraction method, surfactant removal method, or freeze-thaw method. For example, a lipid mixture obtained by adding a desired ratio of lipid nanoparticle material to an organic solvent such as alcohol, and an aqueous buffer solution containing the component to be encapsulated, such as an initializing factor, are prepared, and the aqueous buffer solution is added to the lipid mixture. By agitating and suspending the resulting mixture, lipid nanoparticles that encapsulate the initialization factor, etc., are formed. The lipid nanoparticles obtained in this way are an example of liposomes.

For example, the lipid nanoparticles can be constituted by 60% or less of neutral lipids, cationic lipids, cholesterol, and PEG-modified lipids of the entire component. For example, the lipid nanoparticles may contain more cationic lipids than neutral lipids. Further, the lipid nanoparticles may contain PEG-modified lipids with a maleimide-modified functional group. For example, the lipid nanoparticle may have a lipid composition ratio of FFT-10:FFT-20:cationic lipid:cholesterol:PEG-modified lipid=17 to 23:17 to 23:7 to 13:45 to 51:1.5 to 2.5 (mol %, a total of 100 mol %). With this composition, it is possible to more efficiently initialize blood cell groups.

### Second Embodiment

Such initialization agents may be provided in the form of an introduction carrier set according to the first embodiment described above, ready for use on the desired target cells, or in the form of materials that can be adjusted by the user of the introduction carrier set described above at the time of use.

The second embodiment may be a kit for preparing an introduction carrier or a kit for preparing an initialization agents provided in such a form.

The kit is for initialization of blood cells derived from peripheral blood. The kit comprises a nucleic acid introduction carrier material and an initialization factor group for initialization a group of blood cells including mononuclear cells derived from peripheral blood so as to produce iPS cells. The material of the nucleic acid introduction carrier can be a lipid nanoparticle material for encapsulating the initialization factor group. The lipid nanoparticle material is at least 40% or more of FFT-10 and FFT-20, and FFT-10 and FFT-20 of the entire composition and they are contained in equal amounts.

The kit can be stored in a container in an appropriate state to be provided so that each of the lipid nanoparticle material and the initialization factors can be stably provided as a substance. Here, the initialization factor group may be provided as an initialization factor group itself, or it may be provided such that each factor is contained in a container by the respective type. Prior to use, the kit is constructed as a group of lipid nanoparticles encapsulating the initialization factor group by a means known itself and is provided for use. Further, the kit may also include instructions for preparing the introduction carrier for the user to properly manufacture, for example, a manufacturing manual.

With use of such an introduction carrier preparation kit, it is possible to initialize blood cells derived from peripheral blood. Thus, homogeneously initialized cells, iPS cells can be obtained.

### Third Embodiment

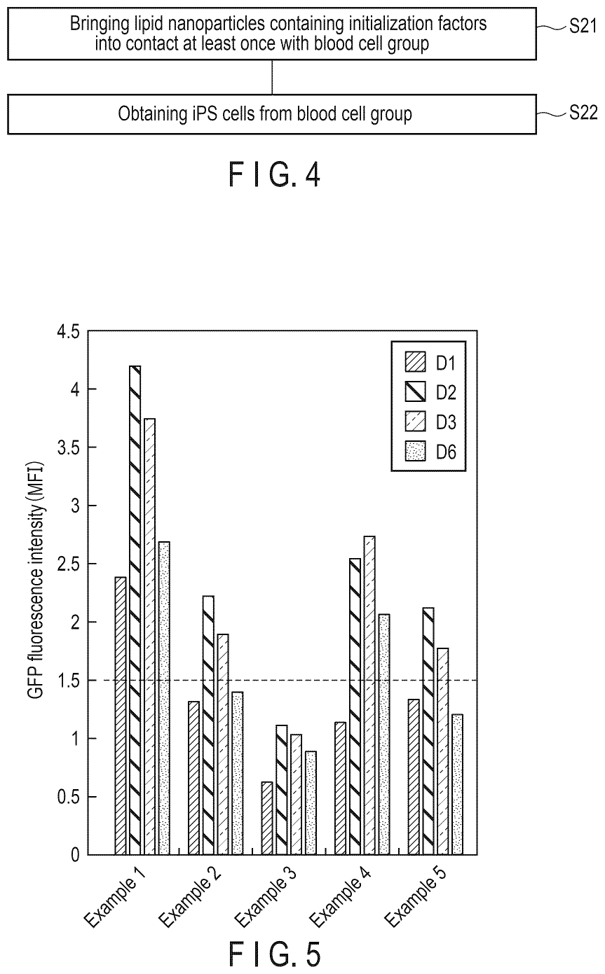

The third embodiment is a method for producing iPS cells by initializing a group of blood cells, including mononuclear cells derived from peripheral blood. As shown in FIG. 4, this method includes bringing the group of blood cells into contact at least once with lipid nanoparticles containing the above-described initialization factors (S21), and obtaining iPS cells from the group of blood cells (S22).

The contact between the lipid nanoparticles encapsulating the initialization factors group and the blood cell group may be carried out at least once, for example, once, twice, three times, four times, five times, or more than once, more than twice, more than three times, more than four times, or once to three times, once to four times, or once to five times.

When the contact is carried out multiple times, the interval between contacts may be, for example, every 12 hours, every 24 hours, every 36 hours, every 48 hours, or a combination of any of these.

The obtaining of iPS cells from a blood cell group can be achieved by bringing the blood cells, which include the initial material cells, that is, mononuclear cells derived from peripheral blood, into contact with the lipid nanoparticles, and thereafter subjecting the resultant to incubation. The expression “obtaining iPS cells” here means that iPS cells are formed from the initial material cells.

The initialization factor group to be introduced may be added to the blood cell group at the same time, or the initialization factors encapsulated in the lipid nanoparticles, which are divided into desired groups, may be added to the blood cell group at the same time or in series.

For example, it may be carried out under an incubation. The incubation conditions may be general conditions suitable for blood cells, for example, 35° C. to 38° C.

The “group of blood cells including mononuclear cells derived from peripheral blood” is referred to the cells to which the initialization factor is to be applied. The blood cell group can be used without individually separating the blood cells from the peripheral blood collected by a general method. For example, the blood cell components from the blood collected from the peripheral blood vessels, and it is possible to use it as an initial material cell group. Substantially, when the mononuclear cells contained in such an initial material cell group are brought into contact with lipid nanoparticles and the initialization factors encapsulated therein act on the cells, iPS cells are prepared. Further, before such an initial material cell group is brought into contact with lipid nanoparticles, the cell group may be cultured for a certain period under conditions that facilitate the proliferation of the hematopoietic stem cells contained within the initial material cell group.

The inventors have been conducting research on the formation of iPS cells by initializing various cells. In such research, as discussed in the embodiments provided above, the inventors have discovered that it is possible to achieve more efficient initialization by using a group of blood cells, including mononuclear cells derived from peripheral blood, as the initial material cell group. The fact that the collection of peripheral blood is minimally invasive is also beneficial, as it places less of a burden on the subject being sampled.

### Examples

Examples of the preparation and use of lipid nanoparticles as a form of lipid nanoparticles of the embodiments will be described.

### Experiment 1: Preparation of Lipid Nanoparticles Encapsulating GFP Gene

As the nucleic acid to be encapsulated in the lipid nanoparticles, the messenger RNA of the green fluorescent protein (GFP) gene was used. The nucleic acid was suspended in 10 mM HEPES (pH 7.3) to obtain a nucleic acid solution.

FFT-10, FFT-20, DOPE, DOTAP, cholesterol and DMG-PEG were dissolved in ethanol at a molar ratio of 0.0:25.8:4.9:9.8:55.8:3.7 to obtain a lipid solution. The lipid solution and the above-mentioned nucleic acid solution were mixed using a microflow chip and syringe pump. After diluting the mixed solution 10-fold with 10 mM HEPES (pH 7.3), the resultant was concentrated using an ultrafiltration filter (Amicon Ultra 0.5 Ultracel-50, Merck), and thus lipid nanoparticles of Examples 1 to 4 were obtained.

TABLE 1
Composition ratio of lipid particle prepared
for lipid nanoparticles (mol %)

| FFT-10 | FFT-20 | DOPE | DOTAP | Chol | PEG |
|---|---|---|---|---|---|
| 0.0 | 25.8 | 4.9 | 9.8 | 55.8 | 3.7 |

### Experiment 2: Quantification of Lipids in Lipid Nanoparticles by LC/MS

In order to compare the composition ratio of lipids used for the preparation of the lipid nanoparticles and the composition ratio of the lipids contained in the lipid nanoparticles prepared, and to evaluate the error range of the lipid composition ratio, lipid was quantified by liquid chromatography mass spectrometry (LC/MS). The lipid nanoparticles prepared in Experiment 1 were diluted with methanol, and using an LC/MS device (ACQUITY UPLC/QTOF System, Waters), FFT-10, FFT-20, DOPE, DOTAP, cholesterol, and DMG-PEG contained in each of the lipid nanoparticles were quantified under the conditions shown in TABLE 2.

TABLE 2

| UPLC | Transfer | 10 mM ammonium acetate | |
|---|---|---|---|
| analysis | phase A | aqueous solution | |
| conditions | Transfer | Methanol | |
| | phase B | | |
| MS | Measurement | Scan positive ion | |
| conditions | mode | | |
| | Ionization | ESI | FFT-10, FFT-20, |
| | mode | | DOPE, DOTAP, DMG-PEG |
| | | APCI | Cholesterol |

The measurements were carried out in accordance with the manual attached with the LC/MS device. The ratio of each of the lipids was converted to molar percentage (mol %) from the lipid quantification results, and the lipid composition ratios of the lipid nanoparticles were calculated. TABLE 1 provided above show the composition ratio of lipids prepared for the lipid nanoparticles, and TABLE 3 and TABLE 4 provided the lipid composition ratio calculated from the results of the LC/MS analysis and the amount of error.

TABLE 3
Lipid composition ratio calculated from
results of LC/MS analysis (mol %)

| | FFT-10 | FFT-20 | DOPE | DOTAP | Chol | PEG |
|---|---|---|---|---|---|---|
| Example | 0.0 | 23.3 | 7.8 | 8.8 | 56.5 | 3.7 |
| 1 | | | | | | |
| Example | 0.0 | 21.8 | 8.5 | 8.6 | 57.4 | 3.7 |
| 2 | | | | | | |
| Example | 0.0 | 23.9 | 7.8 | 9.6 | 55.0 | 3.7 |
| 3 | | | | | | |
| Example | 0.0 | 24.7 | 7.3 | 9.8 | 54.5 | 3.8 |
| 4 | | | | | | |

TABLE 4
Error amount (mol %)

| | | | | | |
|---|---|---|---|---|---|
| FFT-10 | FFT-20 | DOPE | DOTAP | Chol | PEG |

| | | | | | |
|---|---|---|---|---|---|
| Example | −2.5 | 2.9 | −1.0 | 0.7 | 0.0 |
| 1 | | | | | |
| Example | −4.0 | 3.6 | −1.2 | 1.6 | 0.0 |
| 2 | | | | | |
| Example | −2.0 | 2.9 | −0.2 | −0.8 | 0.0 |
| 3 | | | | | |
| Example | −1.2 | 2.4 | 0.0 | −1.4 | 0.1 |
| 4 | | | | | |

The error amounts were calculated using the composition ratio of the lipid prepared as the theoretical value and the lipid composition ratio by LC/MS analysis as the actual measured value. The results indicated that the error range for the above-described lipid nanoparticle preparation method was 0.1 to 4 mol %. When the lipid composition ratio is provided here, an error of 0.1 mol % to 4 mol % may be included as a possibility.

### Experiment 3: Preparation of Lipid Nanoparticles Encapsulating GFP Gene

The messenger RNA of GFP gene was used as the nucleic acid to be encapsulated in the lipid nanoparticles. The nucleic acid was suspended in 10 mM HEPES (pH 7.3) to obtain a nucleic acid solution.

FFT-10, FFT-20, DOPE, DOTAP, cholesterol, and DMG-PEG were dissolved in ethanol each at the composition ratios indicated in TABLE 5 to obtain a lipid solution. The lipid solution and the above-described nucleic acid solution were mixed using a microflow chip and a syringe pump. After diluting the mixed solution 10-fold with 10 mM HEPES (pH 7.3), it was concentrated using an ultrafiltration filter (Amicon Ultra 0.5 Ultracel-50, Merck), and the lipid nanoparticles of Example 5 to Example 9 were obtained, respectively.

TABLE 5

| | Composition ratio of lipid particle prepared (mol %) |
|---|---|
| | (FFT-10:FFT-20:DOPE:DOTAP:Cholesterol:DMG-PEG) |
| Example 5 | 20:20:0:10:48:2 |
| Example 6 | 20:20:10:10:36:4 |
| Example 7 | 58:0:0:17:23:2 |
| Example 8 | 0:32:5:9:51:3 |
| Example 9 | 2:30:2:17:45:4 |

### Experiment 4: Measurement of Intensity of Gene Expression

Human peripheral blood mononuclear cells (PBMC, Precision For Medicine) cultured in StemSpan SFEMII medium (Stemcell technologies) containing six types of cytokines (IL-6, SCF, TPO, Flt-3L, IL-3, and G-CSF; all by Wako) were centrifuged and collected. Then, the resultants were seeded on a 96-well culture plate at 4×10⁴ cells/well (each of the well contains 100 μL of Stem Fit AK03N (with the addition of Rock Inhibitor Y27632)), and each of the lipid nanoparticles prepared in Experiment 3 was added to the respective well by 1.3 μL/well. In each case, the culture plate was placed in an incubator and the cells were cultured at 37° C. in an atmosphere of 5% CO₂.

From the day after the addition of the lipid nanoparticles to the sixth day, the fluorescence intensity of GFP protein expressed from the GFP gene was photographed using a fluorescence microscope (KEYENCE BZ-X810), and the average brightness in each well was measured using image processing software (ImageJ). The measurements were taken according to the manual enclosed with the fluorescence microscope. FIG. 5 shows the results of the measurement of the relative fluorescence intensity of the GFP protein.

Of the five types of lipid nanoparticles (Examples 5 to 9) having different lipid composition ratios of prepared lipids, only the lipid nanoparticles (Example 5) with the composition ratio of FFT-10:FFT-20:DOPE:DOTAP:cholesterol:DMG-PEG=20:20:0:10:48:2 (mol %) showed a relative fluorescence intensity of approximately 2.5 on the first day of measurement (D1), exceeding the reference value of 1.5, and maintained a relative fluorescence intensity of 2.5 or higher until the sixth day of measurement (D6). None of the other lipid nanoparticles (Examples 6 to 9) showed a fluorescence intensity that exceeded the measurement results of Example 5. From the results provided above, it was demonstrated that the lipid nanoparticles composed of the lipid composition ratio of Example 5 can deliver nucleic acids to PBMCs cultured under the above conditions at high efficiency.

### Experiment 5: Preparation of Lipid Nanoparticles Encapsulating Initialization Factor Group

The messenger RNA of six types of initialization factors (Oct3/4, Sox2, Klf4, cMyc, Nanog, Lin28) was used as the nucleic acid to be encapsulated in the lipid nanoparticles. The nucleic acids were suspended in 10 mM HEPES (pH 7.3) to obtain a nucleic acid solution. The lipid solutions were prepared by dissolving FFT-10, FFT-20, DOPE, DOTAP, cholesterol, and DMG-PEG in ethanol at a molar ratio of 20:20:0:10:48:2, respectively. The lipid solution and the above-mentioned nucleic acid solution were mixed using a microflow chip and a syringe pump. After diluting the mixed solution 10-fold with 10 mM HEPES (pH 7.3), the mixture was concentrated using an ultrafiltration filter (Amicon Ultra 0.5 Ultracel-50, Merck) to obtain the lipid nanoparticles of Example 10.

### Experiment 6: Cell Initialization Test

Human peripheral blood mononuclear cells (PBMC, Precision For Medicine) cultured in StemSpan SFEMII medium (Stemcell technologies) containing six types of cytokines (IL-6, SCF, TPO, Flt-3L, IL-3, and G-CSF; all by Wako) were centrifuged and collected. Then, the resultants were seeded on a 24-well culture plate at 2×10⁵ cells/well (each of the well contains 600 μL/well of Stem Fit AK03N (with the addition of Rock Inhibitor Y27632)), and each of the lipid nanoparticles prepared in Experiment 5 was added to each of the well under the conditions indicated in TABLE 6. In each case, the culture plate was placed in an incubator and the cells were cultured at 37° C. in an atmosphere of 5% CO₂.

TABLE 6

| | Day 0 | Day 1 | Day 2 | Day 3 | Day 4 | Day 5 |
|---|---|---|---|---|---|---|
| Condition | 2 μL/ | 2 μL/ | 2 μL/ | 2 μL/ | 2 μL/ | 2 μL/ |
| 1 | well | well | well | well | well | well |
| Condition | 4 μL/ | — | 4 μL/ | — | 2 μL/ | — |
| 2 | well | | well | | well | |
| Condition | 4 μL/ | — | — | — | — | — |
| 3 | well | | | | | |

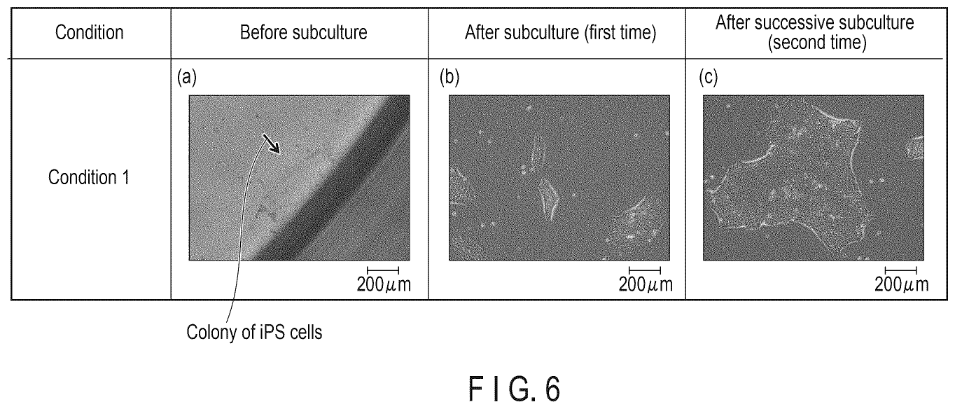

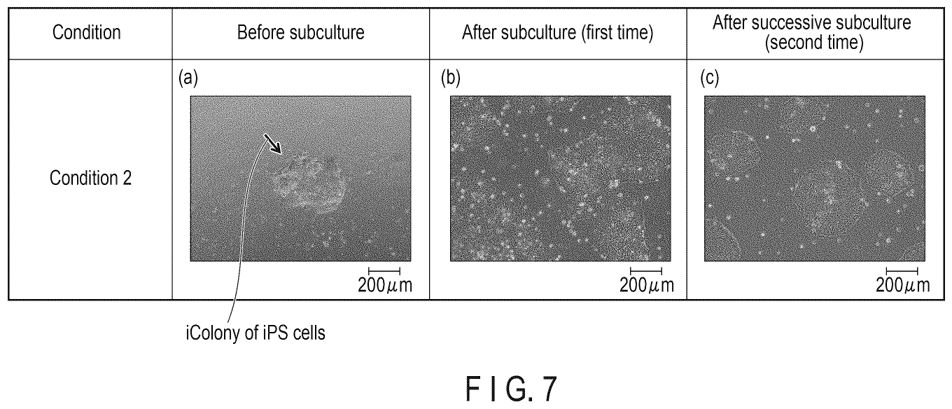

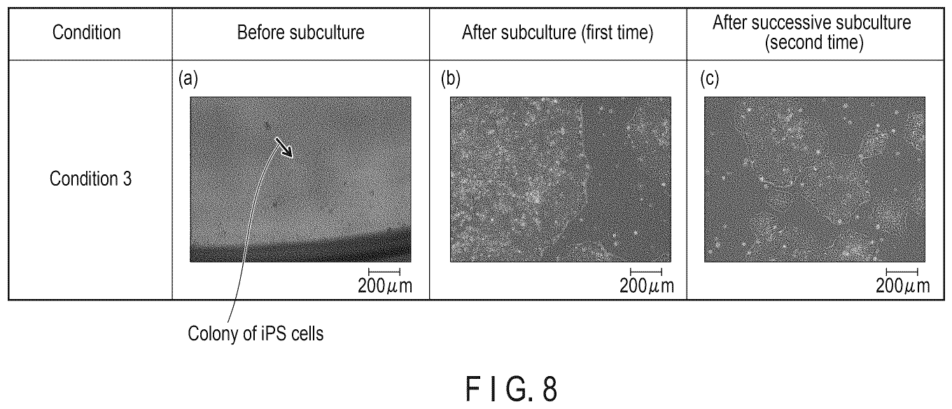

The cell culture was continued while adding culture medium every 3 to 4 days, and whether colonies of iPS cells were formed was observed under an optical microscope. FIGS. 6 to 8 show the images of the cells observed under the optical microscope before subculture, after the subculture (the first time), and after a successive subculture (the second time), for Conditions 1 to 3, respectively. Further, the cells from Condition 3 were collected and Alexa 647-labeled TRA-1-60 antibody (R&D systems) was added thereto, and then the expression of the pluripotent stem cell marker TRA-1-60 was measured using a flow cytometer (Sony SA3800). The measurements were carried out in accordance with the manual supplied with the flow cytometer. TABLE 7 shows the percentage of viable cells and TRA-1-60 expression rate as measured by the flow cytometer.

TABLE 7

| | Viable | TRA-1-60 |
|---|---|---|
| | cell ratio | expression rate |
| Condition 3 | 76% | 93% |

In each of all three conditions, where the timing of addition and the amount of lipid nanoparticles were varied, it was observed that the image of colonies of iPS cells had been formed before the subculture (approximately 10 days after the addition of lipid nanoparticles). Further, the formation of colonies of iPS cells was observed after the second subculture (approximately 24 days after) as well. The iPS cells that formed the colonies were collected and the expression of the initialization marker, TRA-1-60 was measured using a flow cytometer. The results indicated that an expression rate of 96% was obtained for TRA-1-60 under Condition 3 (that is, cells cultured with lipid nanoparticles added once on the first day).

From the results obtained as above, it has been confirmed that with the lipid nanoparticles and the methods according to the embodiments, it is possible to provide a technology for producing iPS cells by initializing a group of blood cells, including mononuclear cells derived from peripheral blood.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

The followings are descriptions of examples of further embodiments.
(1) An agent for initializing a group of blood cells, comprising a initialization factor group for producing iPS cells by initializing a group of blood cells including mononuclear cells derived from peripheral blood, and a group of lipid nanoparticles encapsulating the initialization factor group, wherein a lipid component of the lipid nanoparticles contains at least 40% or more of FFT-10 and FFT-20, and FFT-10 and FFT-20 are contained in equal amounts.
(2) The agent according to clause (1), wherein a component of the lipid nanoparticles contains 60% or less of neutral lipids, cationic lipids, cholesterol, and PEG-modified lipids.
(3) The agent according to clause (1) or (2), wherein the lipid nanoparticles contain more cationic lipids than neutral lipids.
(4) The agent according to any one of clauses (1) to (3), wherein the lipid nanoparticles are PEG-modified lipids having a maleimide-modified functional group.
(5) The agent according to any one of clauses (1) to (4), wherein the lipid nanoparticles have a lipid composition ratio of FFT-10:FFT-20:cationiclipid:cholesterol:PEG-modified lipid=17 to 23%:17 to 23%:7 to 13%:45 to 51%:1.5 to 2.5% in mol %, and a value of each component is selected to made a total of 100 mol %.
(6) The agent according to any one of clauses (1) to (5), wherein the initialization factor group encapsulated in the lipid nanoparticles includes Oct3/4, Sox2, Klf4, and cMyc.
(7) The agent according to any one of clauses (1) to (6), wherein the initialization factor group encapsulated in the lipid nanoparticles further includes Nanog and Lin28.
(8) The agent according to any one of clauses (1) to (7), wherein the initialization factor group encapsulated in the lipid nanoparticles is DNA, RNA and/or protein.
(9) The agent according to any one of clauses (1) to (8), wherein the lipid nanoparticles contain a further factor other than the initialization factor group.
(10) The agent according to any one of clauses (1) to (9), wherein the lipid nanoparticles further contain a factor that suppresses response of interferon as an encapsulated component other than the initialization factor group.
(11) The agent according to any one of clauses (1) to (10), wherein the lipid nanoparticles further contain miRNA that improves efficiency of establishing iPS cells as an encapsulated component other than the initialization factor group.
(12) The agent according to any one of clauses (1) to (11), wherein encapsulation of the initialization factor group into the lipid nanoparticle group is such that all of the initialization factor group is encapsulated together per particle, or the initialization factor group is divided into multiple lipid nanoparticles and encapsulated therein.
(13) A kit of preparing an agent for initializing a group of blood cells, comprising a initialization factor group for producing iPS cells by initializing a group of blood cells including mononuclear cells derived from peripheral blood, and a lipid nanoparticle material for encapsulating the initialization factor group, wherein a lipid component of the lipid nanoparticle material contains at least 40% or more of FFT-10 and FFT-20, and FFT-10 and FFT-20 are contained in equal amounts.
(14) The kit according to clause (13), wherein a component of the lipid nanoparticle material contains 60% or less of neutral lipids, cationic lipids, cholesterol, and PEG-modified lipids.
(15) The kit according to clause (13) or (14), wherein the lipid nanoparticle material contains more cationic lipids than neutral lipids.
(16) The kit according to any one of clauses (13) to (15), wherein the lipid nanoparticle material is PEG-modified lipids having a maleimide-modified functional group.
(17) The kit according to any one of clauses (13) to (16), wherein the lipid nanoparticle material has a lipid composition ratio of FFT-10:FFT-20:cationiclipid:cholesterol:PEG-modified lipid=17 to 23%:17 to 23%:7 to 13%:45 to 51%:1.5 to 2.5% in mol %, and a value of each component is selected to made a total of 100 mol %.
(18) The kit according to any one of clauses (13) to (17), wherein the initialization factor group includes Oct3/4, Sox2, Klf4, and cMyc.
(19) The kit according to clause (18), wherein the initialization factor group further includes Nanog and Lin28.
(20) The kit according to any one of clauses (13) to (19), wherein the initialization factor group is DNA, RNA and/or protein.
(21) The kit according to any one of clauses (13) to (20), containing a further factor other than the initialization factor group.
(22) The kit according to any one of clauses (13) to (21), further containing a factor that suppresses response of interferon other than the initialization factor group.
(23) The kit according to any one of clauses (13) to (22), further containing miRNA that improves efficiency of establishing iPS cells other than the initialization factor group.
(24) The kit according to any one of clauses (13) to (23), further comprising a manufacturing manual.
(25) A method for producing iPS cells from a blood cell group, comprising bringing an initialization agent according to any one clauses (1) to (12) into contact with a blood cell group including mononuclear cells derived from blood, and obtaining iPS cells from the blood cell group.

What is claimed is:

1. An agent for initializing a group of blood cells, comprising a initialization factor group for producing iPS cells by initializing a group of blood cells including mononuclear cells derived from peripheral blood, and a group of lipid nanoparticles encapsulating the initialization factor group, wherein a lipid component of the lipid nanoparticles contains at least 40% or more of FFT-10 and FFT-20, and FFT-10 and FFT-20 are contained in equal amounts.

2. The agent of claim 1, wherein a component of the lipid nanoparticles contains 60% or less of neutral lipids, cationic lipids, cholesterol, and PEG-modified lipids.

3. The agent of claim 1, wherein the lipid nanoparticles contain more cationic lipids than neutral lipids.

4. The agent of claim 2, wherein the lipid nanoparticles are the PEG-modified lipids having a maleimide-modified functional group.

5. The agent of claim 1, wherein the lipid nanoparticles have a lipid composition ratio of FFT-10:FFT-20:cationic lipid:cholesterol:PEG-modified lipid=17 to 23%:17 to 23%:7 to 13%:45 to 51%:1.5 to 2.5% in mol %, and a value of each component is selected to made a total of 100 mol %.

6. The agent of claim 1, wherein the initialization factor group encapsulated in the lipid nanoparticles includes Oct3/4, Sox2, Klf4, and cMyc.

7. The agent of claim 6, wherein the initialization factor group encapsulated in the lipid nanoparticles further includes Nanog and Lin28.

8. The agent of claim 1, wherein the initialization factor group encapsulated in the lipid nanoparticles is in a form of DNA, RNA and/or protein.

9. The agent of claim 1, wherein the lipid nanoparticles contain a further factor other than the initialization factor group.

10. The agent of claim 1, wherein the lipid nanoparticles further contain a factor that suppresses response of interferon as an encapsulated component other than the initialization factor group.

11. The agent of claim 1, wherein the lipid nanoparticles further contain miRNA that improves efficiency of establishing iPS cells as an encapsulated component other than the initialization factor group.

12. The agent of claim 1, wherein encapsulation of the initialization factor group into the lipid nanoparticle group is such that all of the initialization factor group is encapsulated together per particle, or the initialization factor group is divided into multiple lipid nanoparticles and encapsulated therein.

13. A kit of preparing an agent for initializing a group of blood cells, comprising a initialization factor group for producing iPS cells by initializing a group of blood cells including mononuclear cells derived from peripheral blood, and a lipid nanoparticle material for encapsulating the initialization factor group, wherein a lipid component of the lipid nanoparticle material contains at least 40% or more of FFT-10 and FFT-20, and FFT-10 and FFT-20 are contained in equal amounts.

14. The kit of claim 13, wherein a component of the lipid nanoparticle material contains 60% or less of neutral lipids, cationic lipids, cholesterol, and PEG-modified lipids.

15. The kit of claim 13, wherein the lipid nanoparticle material contains more cationic lipids than neutral lipids.

16. The kit of claim 14, wherein the lipid nanoparticle material is the PEG-modified lipids having a maleimide-modified functional group.

17. The kit of claim 13, wherein the lipid nanoparticle material has a lipid composition ratio of FFT-10:FFT-20:cationic lipid:cholesterol:PEG-modified lipid=17 to 23%:17 to 23%:7 to 13%:45 to 51%:1.5 to 2.5% in mol %, and a value of each component is selected to made a total of 100 mol %.

18. The kit of claim 13, wherein the initialization factor group includes Oct3/4, Sox2, Klf4, and cMyc.

19. The kit of claim 18, wherein the initialization factor group further includes Nanog and Lin28.

20. A method for producing iPS cells from a blood cell group, comprising bringing an initialization agent of claim 1 into contact with a blood cell group including mononuclear cells derived from blood, and obtaining iPS cells from the blood cell group.
